(19) United States
(12) Patent Application Publication
(10) Pub. No.: US 2004/0265337 A1
(43) Pub. Date: Dec. 30, 2004

(54) Method of generating an immune response and compositions used for same

(76) Inventors: Krisztina M. Zsebo, La Jolla, CA (US); Roymarie Ballester, Santa Barbara, CA (US); Gary Schoolnik, Palo Alto, CA (US); Steven M. Julio, Santa Barbara, CA (US); Andrew F. Giusti, San Diego, CA (US)

Correspondence Address: MORRISON & FOERSTER LLP, 755 PAGE MILL RD, PALO ALTO, CA, 94304-1018, US

(21) Appl. No.: 10/646,948

(22) Filed: Aug. 21, 2003

Related U.S. Application Data
(60) Provisional application No. 60/405,603, filed on Aug. 21, 2002.

(51) Int. Cl. A61K 39/02

(52) U.S. Cl. 424/200.1

(57) ABSTRACT

This invention provides immunogenic compositions containing attenuated bacteria (such as Salmonella enterica) which are resistant to the antimicrobial actions of human defensins, particularly human defensin 5 (HD-5). Methods for using these compositions to elicit a sustained and highly specific immune response are provided. The invention also provides methods for preparing vaccines wherein a heterologous antigen is expressed by the defensin-resistant bacteria.

### CROSS-REFERENCE TO RELATED APPLICATIONS

This patent application claims priority to U.S. provisional patent application Ser. No. 60/405,603, filed Aug. 21, 2002, which is incorporated herein by reference in its entirety.

### FIELD OF THE INVENTION

The present invention relates generally to methods of creating an immune response and to compositions including vaccines used in the methods. In particular, this invention relates to methods of creating immune response using immunogenic compositions generally comprising formulations of bacteria which are normally pathogenic bacteria (e.g., Salmonella enterica) which are resistant to the actions of human defensins, particularly human defensin 5 (HD-5).

### BACKGROUND OF THE INVENTION

Whole bacterial vaccines such as Salmonella enterica can be used as a delivery vehicle and concomitant adjuvant in vaccine formulations. The wild-type organisms cause diseases that include acute gastroenteritis and enteric fevers. Salmonella infections are generally acquired by oral ingestion. The microorganisms after traversing the stomach, invade and replicate in the intestinal mucosal cells. See, Homik, et al., N. Eng. J. Med., 283:686 (1970). Salmonella enterica serovars target a major immunological organ after oral ingestion by passing through this mucosal barrier and spreading via the Peyer's patches to the lamina propia and regional lymph nodes. Therefore, vaccine compositions of highly attenuated strains of Salmonella enterica, which have altered genes rendering them non-pathogenic or attenuated, can be used as delivery vehicles for foreign antigens and DNA, and induce an immune response in a mammal. See, Shata et. al., Molecular Medicine Today, 6: 66 (2000). Vaccination, at least in parts of the world, has controlled the following nine major diseases: smallpox, diphtheria, tetanus, yellow fever, pertussis, poliomyelitis, measles, mumps and rubella. In the case of smallpox, the disease has been totally eradicated from the world. Cancer is also now being treated with vaccines, where the vaccines elicit an immune response against tumor antigens. The effectiveness of a vaccine depends upon its ability to elicit a protective immune response against specific molecular structures contained in the vaccine preparation, or antigens which will be generally described below.

A well-characterized mechanism of adaptive immune response to foreign antigens is the activation of T and B cells by the host. The cellular immune response is driven primarily by T cells, which generally recognize pathogens which are intra-cellular (i.e., exist for a portion of their life cycle inside the mammalian cell) or tumor cells. B cells generally recognize antigens which exist for a period outside the mammalian cell, and often circulate in the blood. Antigen specific T cells are stimulated by recognizing antigen presented by cells such as macrophages and dendritic cells. Macrophages and dendritic cells are potent antigen presenting cells (APC's), and have a variety of receptors that recognize microbial constituents such as lipopolysaccharide. These receptors bind microorganisms and the macrophage engulfs them and degrades the microorganisms in the endosomes and lysosomes. The antigens are taken up and processed by APC's and re-presented via class I and class II HLA receptors to T cells. B cells recognize antigens initially by B cell associated IgM antibody binding to extra-cellular structures, either freely circulating in the blood, or on the surface of organisms or tumor cells. The B cell then develops to produce other forms of antigen specific antibody, such as IgG or IgA, which are secreted into the blood. Following the first exposure to an antigen the immune response is often slow and the affinity of T cells or antibody produced is weak, i.e., the primary response. On secondary challenge with the same antigen, the response, i.e., the secondary response, is more rapid and of higher affinity thereby achieving effective eradication or control of the pathogen or tumor cell, which is the goal that is sought to be induced by vaccines.

In general, active vaccines can be divided into two general classes: subunit vaccines and whole organism vaccines. Subunit vaccines are prepared from components of the whole organism or tumor cell. The use of purified capsular polysaccharide material of H. influenza type b as a vaccine against bacterial meningitis in humans is an example of a vaccine based upon an antigenic component. See Parks, et al., J. Inf. Dis., 136 (Suppl.):551 (1977); Anderson, et al., J. lnf. Dis., 136 (Suppl.):563 (1977); and Mäkela, et al., J. Inf. Dis., 136 (Suppl.):543 (1977).

Classically, subunit vaccines have been prepared by chemical inactivation of partially purified toxins, and hence have been called toxoids. Formaldehyde or glutaraldehyde have been the chemicals of choice to detoxify bacterial toxins. Both diphtheria and tetanus toxins have been successfully inactivated with formaldehyde resulting in a safe and effective toxoid vaccine which has been used for over 40 years to control diphtheria and tetanus. See, Pappenheimer, A. M., Diphtheria. In: Bacterial Vaccines (R. Germanier, ed.), Academic Press, Orlando, Fla., pp. 1-36 (1984); Bizzini, B., Tetanus. Id. at 37-68. Chemical toxoids, however, are not without undesirable properties. In fact, this type of vaccine can be more difficult to develop since protective antigens must first be identified and then procedures must be developed to efficiently isolate the antigens. Furthermore, in some cases, subunit vaccines do not elicit as strong an immune response as do whole organism vaccines due to the lack of extraneous materials such as membranes or endotoxins. These structures are recognized by APC's as a signal of an invading pathogen, and result in a strong signal (cytokines and co-stimulatory molecules) by APC's to T cells and B cells, which when present, subsequently mount an effective immune response.

Whole organism vaccines, on the other hand, make use of the entire organism for vaccination. The organism may be killed or alive (usually attenuated) depending upon the requirements to elicit protective immunity. The pertussis vaccine, for example, is a killed whole cell vaccine prepared by treatment of Bordetella pertussis cells with formaldehyde. The bacterium B. pertussis colonizes the epithelial lining of the respiratory tract resulting in a highly contagious respiratory disease in humans, pertussis or whooping cough, with morbidity and mortality rates highest for infants and young children. The colonization further results in local tissue damage and systemic effects caused in large part by toxins produced by B. pertussis. See, Manclarck, et al., Pertussis., Id. at 64-106. These toxins include endotoxin or lipopolysaccharide, a peptidoglycan fragment called tracheal cytotoxin, a heat-labile dermonecrotizing protein toxin, an adenylate cyclase toxin, and the protein exotoxin pertussis toxin. Vaccination is the most effective method for controlling pertussis, and killed whole-cell vaccines administered with diphtheria and tetanus toxoids (DPT vaccine) have been effective in controlling disease in many countries. See, Fine, et al., Reflections on the Efficacy of Pertussis Vaccines, Rev. Infect. Dis., 9:866-883 (1987). Unfortunately, due to the large amounts of endogenous products, discussed above, contained in the pertussis vaccine, many children experience adverse reactions upon injection. Endotoxin, which is an integral component of the outer membrane of this gram-negative organism (as well as all other gram-negative organisms), can induce a wide range of mild to severe side effects including fever, shock, leukocytosis, and spontaneous abortion. While the side effects associated with pertussis vaccine are usually mild, they may be quite severe. The toxic components present in influenza virus vaccines, however, can induce a strong pyrogenic response and have been responsible for the production of Guillain-Barré syndrome. Since influenza vaccines are prepared by growth of the virus in chick embryos, it is likely that components of the embryo or egg contributes to this toxicity.

The use of killed vaccines has also been described by Switzer et al., U.S. Pat. No. 4,016,253, who applied such a method in preparing a vaccine against Bordetella bronchiseptica infection in swine. In a technical paper by Brown, et al., Br. Med. J., 1:263 (1959), the use of killed whole cells is disclosed for preparing a vaccine against chronic bronchitis caused by Haemophilus influenzae. The use of killed cells, however, is usually accompanied by an attendant loss of immunogenic potential, since the process of killing usually destroys or alters many of the surface antigenic determinants necessary for induction of specific antibodies in the host. Killed vaccines are ineffective or marginally effective for a number of pathogenic bacteria including Salmonella spp. and V. cholerae. The parenteral killed whole cell vaccine now in use for Salmonella typhi is only moderately effective, and causes marked systemic and local adverse reactions at an unacceptably high frequency.

These microorganisms can also be designed to express a particular protein (heterologous protein) or deliver DNA to mammalian cells that will be expressed into protein while in the host cell, and subsequently stimulate an immune response (T cells, B cells, or both) against the heterologous antigen. Different bacteria have been used in this regard. Salmonella enterica vaccines are attractive vaccine vehicles, since they are orally available, stimulate a strong cellular immune response, and have a good safety profile. Salmonella enterica vaccines have been very effective in rodent models to stimulate an immune response against heterologous antigens. See, Eisenstein (1999) Intracellular Bacterial Vaccine Vectors (Paterson, ed., Wiley-Liss, Inc.) pp. 51-109; Hone et al. Intracellular Bacterial Vaccine Vectors (Paterson, ed., Wiley-Liss, Inc.) pp. 171-221 (1999); Sirard et al. Immnun. Rev. 171:5-26 (1999); Shata et. al., Molecular Medicine Today, 6: 66 (2000). However, these results have not been reproduced in human clinical studies. See, DiPetrillo et. al, Vaccine 18: 449 (2000). The subject of this application are vaccine compositions which enhance the effectiveness of whole organism vaccines in higher species (greater than rodents), or in human defensin transgenic mice.

Mice and humans differ in their susceptibility to Salmonella enterica serovar Typhimurium infections. One major difference between humans and mice affecting susceptibility is the expression of specific anti-microbial peptides, called defensins in humans and cryptdins in mice, in the intestine. Human defensin 5 (HD-5) is the major defensin in humans which is bactericidal for Salmonella enterica serovar Typhimurium infection. See, Ghosh D, et. al., Nature Immunology,3:583 (2002). Mice do not contain HD-5, instead they contain cryptdins, which are less active against Salmonella enterica than HD-5. See Wilson et. al., Science 286:113 (1999). Mice transgenic for HD-5 are resistant to Salmonella enterica serovar Typhimurium infection. See, Zasloff, Nature Immunology, 3:508 (2002).

By appreciating the effects of HD-5 we have deduced that the expression in humans (but not in mice) of HD-5 makes humans less susceptible to Salmonella enterica serovar Typhimurium infections than mice resulting in a lack of translation of efficacy of Salmonella enterica based vaccines from mice to humans. More specifically, the greater bactericidal activity of HD-5 in the intestine of humans modulated the human immune response from vaccines making those vaccines less efficacious in humans as compared to mice.

All references and patent applications cited within this application are herein incorporated by reference in their entirety.

### SUMMARY OF THE INVENTION

Human defensin-5 (HD-5) is a barrier to effective vaccination of higher species, such as primates and humans as well as human defensin transgenic mice, against antigens carried by whole organism vaccines such as Salmonella enterica serovars, particularly Typhimurium. Vaccine compositions of the invention (1) render the bacteria resistant to the anti-microbial action of human defensins, in particular HD-5, and (2) are more effective in eliciting an immune response in a higher species, such as a primate or human subject, or human defensin transgenic mice which generates antigen-specific T cells, B cells, or both, which can be measured.

An aspect of the invention is a composition of whole organism vaccines such as Salmonella enterica serovars, particularly Typhimurium, which render attenuated vaccine bacteria in the composition resistant to the anti-microbial action of human defensins.

Another aspect of the invention is using altered bacteria to produce T cells and B cells (antibodies), which are highly specific to the bacteria, or to heterologous antigens carried by the bacteria.

Another aspect of the invention is to provide live vaccines which serve as carriers for antigens, preferably immunogens, such as tumor cells or microorganisms, including viruses, prokaryotes, and eukaryotes.

In another aspect, the invention provides methods of eliciting an immune response in an individual comprising administering any of the compositions described herein (including any of the strains described herein) to an individual (e.g. a human) in an amount sufficient to elicit an immune response.

Another aspect of the invention provides a method of treating cancer or infection by pathogens in an individual, comprising administering to a human patient in need of treatment an immunogenic composition of the invention to the individual in an amount sufficient to reduce (or ameliorate) a symptom associated with an infectious disease or cancer.

The invention also provides methods of preparing vaccines, strains, and formulations described herein. In one aspect, the invention provides methods of preparing the immunogenic compositions described herein, comprising combining a pharmaceutically excipient with pathogenic bacteria which increases the resistance to the antimicrobial action of human defensins.

Additional objects, advantages and novel features of this invention shall be set forth in part in the description that follows, and in part will become apparent to those skilled in the art upon examination of the following specification or may be learned by the practice of the invention. The objects and advantages of the invention may be realized and attained by means of the instrumentalities, combinations, compositions, and methods particularly pointed out in the appended claims.

These and other objects, advantages, and features of the invention will become apparent to those persons skilled in the art upon reading the details of the invention as more -fully described below.

### BRIEF DESCRIPTIONS OF THE DRAWINGS

FIG. 1 is an amino acid sequence of amino acids 1-19 of the signal peptide of HD-5.

FIG. 2 is an amino acid sequence of amino acids 20-62 showing the pro-piece of HD-5.

FIG. 3 is the amino acid sequence showing amino acids 63-94 of mature HD-5.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a bacteria” includes a plurality of such bacteria and reference to “the mutation” includes reference to one or more mutations and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The terms “treat,” “treatment,” and the like are used herein to generally mean a desired pharmacological and/or physiological effect. The effect may be prophylactic in terms of completely or partially preventing a disease or infection and/or may be therapeutic in terms of partially or completely curing the disease or infection and/or adverse effect attributed to the disease or infection. In general, methods of the invention involve treating infectious diseases associated with infections from bacteria or viruses and further includes treatment due to infection with any pathogen or the presence of a cancer. “Treatment” as used herein covers any treatment of such a symptom, disease or infection in a mammal, particularly a human, and includes: (a) preventing or diagnosing the disease, infection or symptom in the subject which may be predisposed to the disease, infection and/or symptom but has not yet been diagnosed as having it; (b) inhibiting the disease or infection, i.e. arresting it's development; and/or (c) relieving the disease, infection and/or symptom, i.e. causing regression of the disease, infection and/or symptom caused by the disease or infection.

The invention is directed towards modulating the effect of defensins and in particular modulating the effect of HD-5. By modulating the effect of defensins such as HD-5 a live attenuated bacterial vaccine is not disrupted by the defensin thereby allowing antigens delivered by the bacteria or on the surface of the bacteria to be presented to the immune system thereby allowing the immune system to generate antibodies and induce T-cell responses to those antigens. Antibodies and cytotoxic T cells are then effective in binding to actual antigen expressing cell or pathogenic infectious bacteria which ultimately results in their destruction. Treatments of the invention can be carried out in a variety of different ways using a variety of different mechanisms of action and the treatment may be combined with other treatments including the use of other vaccines, antibodies and/or antibacterial agents.

“Treatment” is an approach for obtaining beneficial or desired clinical results. Beneficial or desired clinical results include, but are not limited to, alleviation of symptoms, diminishment of extent of disease, stabilized (i.e., not worsening) state of disease, preventing the disease or the spread of disease, delay or slowing of disease progression, amelioration or palliation of the disease state.

The term “defensin” as used herein refers to a protein present within a mammal such as human which protein is a non-antibody protein and which protein plays a role in the destruction of foreign substances such as infectious bacteria and/or infectious viruses. As an example HD-5 is a defensin which plays a role in the destruction of a bacterial infection and in particular the destruction of pathogenic Salmonella in a human.

A “vaccine” is a pharmaceutical composition for human or animal use, particularly an immunogenic composition which is administered with the intention of conferring the recipient with a degree of specific immunological reactivity against a particular target, or group of targets (i.e., elicit and/or enhance an immune response against a particular target or group of targets). The immunological reactivity, or response, may be antibodies or cells (particularly B cells, plasma cells, T helper cells, and cytotoxic T lymphocytes, and their precursors) that are immunologically reactive against the target, which is a heterologous antigen. The immunological reactivity may be desired for experimental purposes, for treatment of a particular condition, for the elimination of a particular substance, and/or for prophylaxis.

“Attenuated” bacteria used in the compositions described herein are bacteria which exhibit reduced virulence. As is well understood in the art, and described above, virulence is the degree to which bacteria are able to cause disease in a given population. For purposes of the invention, attenuated bacteria have virulence reduced to a suitable and acceptable safety level, as is generally dictated by appropriate government agencies. The degree of attenuation which is acceptable depends on, inter alia, the recipient (i.e., human or non-human) as well as various regulations and standards which are provided by regulatory agencies such as the U.S. Food and Drug Administration (FDA). Most preferably, especially for human use, attenuated bacteria are avirulent, meaning that administration of these organisms cause no disease symptoms. As is well understood in the art, attenuated bacteria are alive, at least at the time of administration.

“Antigen” means a substance that is recognized and bound specifically by an antibody or by a T cell antigen receptor. As is well understood in the art, antigens can include peptides, proteins, glycoproteins, polysaccharides, gangliosides and lipids, as well as portions and/or combinations thereof. Antigens can be those found in nature or can be synthetic.

An “adjuvant” is a chemical or biological agent given an antigen (e.g. given in combination with an attenuated bacteria as described herein) to enhance its immunogenicity. As is known in the art, an “adjuvant” is a substance which, when added to an antigen, nonspecifically enhances or potentiates an immune response to the antigen in the recipient (host).

“Stimulating”, “eliciting”, or “provoking” an immune response (which can be a B and/or T cell response) means an increase in the response, which can arise from eliciting and/or enhancement of a response.

“Heterologous” means derived from and/or different from an entity to which it is being compared. For example, a “heterologous” antigen with respect to a bacterial strain is an antigen which is not normally or naturally associated with that strain.

An “effective amount” is an amount sufficient to effect a beneficial or desired result including a clinical result, and as such, an “effective amount” depends on the context in which it is being applied. An effective amount can be administered in one or more doses. For purposes of this invention, an effective amount of a vaccine (e.g. a bacterial composition resistant to the antimicrobial action of human defensins) is an amount that induces an immune response. In terms of treatment, an effective amount is amount that is sufficient to palliate, ameliorate, stabilize, reverse or slow the progression of a bacterial disease, or otherwise reduce the pathological consequences of the disease. In terms of prevention, an effective amount is an amount sufficient to reduce (or even eliminate) one or more symptoms upon exposure and infection.

“Preventing” disease or infection is part of treating and specifically means a reduction (including, but not limited to, elimination) of one or more symptoms of infection in an individual receiving a composition described herein as compared to otherwise same conditions except for receiving the composition(s). As understood in the art, “prevention” of a disease or infection can include milder symptoms and does not necessarily mean elimination of symptoms associated with infection.

An “individual”, used interchangeably with “host”, is a vertebrate, preferably a mammal, more preferably a human. Mammals include, but are not limited to, farm animals (such as cattle), sport animals, and pets. An “individual” also includes fowl, such as chickens. A “host” may or may not have been infected with a bacteria.

An “agent” means a biological or chemical compound such as a simple or complex organic or inorganic molecule, a polypeptide, a polynucleotide, carbohydrate or lipoprotein. As vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. Compounds can be tested singly or in combination with one another.

“Comprising” and its cognates mean “including”.

“A”, “an” and “the” include plural references, unless otherwise indicated. For example, “a” defensin means any one or more defensin molecules.

Invention in General

Vaccines of the invention comprise attenuated, whole organism vaccines such as Salmonella enterica, particularly serovar Typhimurium, which are resistant to the antimicrobial effects of human defensins thereby making the attenuated vaccine more effective in inducing an immune response against heterologous antigens in higher species, e.g. humans, or human defensin transgenic mice compared to such vaccines which are not resistant to defensins. The present invention is directed towards: (i) vaccines compositions containing human defensin inhibitors which are expressed in the attenuated vaccine bacteria themselves; (ii) vaccine compositions of bacteria which are co-formulated with inhibitors of human defensins; and (iii) vaccine compositions of bacteria which are co-formulated with inhibitors of human pro-defensin processing.

Methods of eliciting an immune response using the immunogenic compositions described herein include methods for treating higher species such as primates or humans or human defensin transgenic mice with (i) the vaccines of the present invention or (ii) methods of preventing or curing an infection or cancer using the immunogenic compositions described herein.

The ability of certain vaccines (e.g. attenuated whole organism vaccines) to generate an immune response in a human can be substantially enhanced by inhibiting the antimicrobial action of human defensins. This is particularly effective when the defensin inhibitor blocks the action or production of mature HD-5 and the attenuated bacterial vaccine is attenuated Salmonella enterica, particularly serovar Typhimurium.

The immune response generated is to a heterologous antigen which may be (1) an antigen of a pathogenic virus; (2) an antigen of a pathogenic bacteria; (3) an antigen of a pathogenic parasite, (4) an antigen of a pathogenic fungus, (5) an antigen of a mammalian tumor; and/or (6) an antigen is a mammalian immune disease.

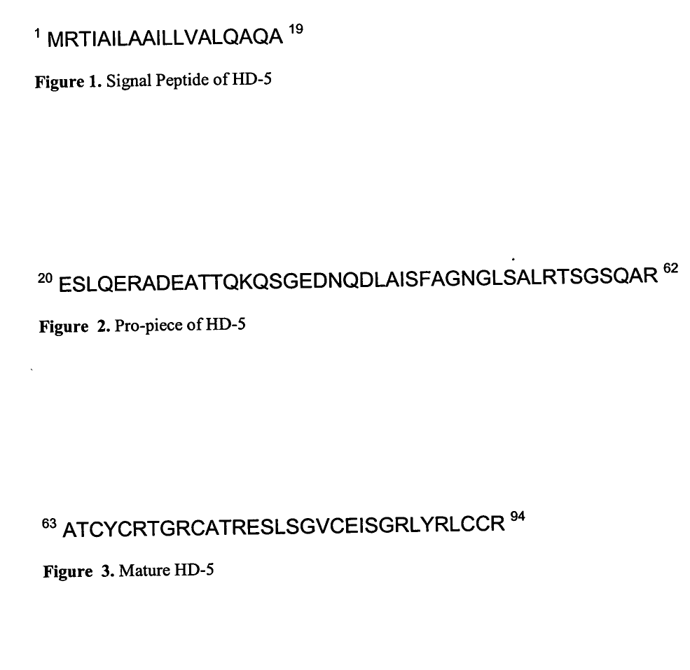

The amino acid sequences of a number of defensins such as HD-5 are known. The pre-propeptide of HD-5 comprises 94 amino acids (see FIGS. 1-3; SEQ ID NO: 1) and is processed to the pro-peptide comprised of amino acids 20-94 (see FIGS. 2 and 3; SEQ ID NO: 2) by the removal of the signal peptide comprising amino acids 1-19 (see FIG. 1; SEQ ID NO: 3). The pro-peptide is stored in the granules of Paneth cells in the intestine of humans. Upon degranulation the pro-HD-5 peptide is processed by protease digestion in order to generate the mature HD-5 protein comprised of amino acids 63-94 (see FIG. 3; SEQ ID NO: 4). The amino acid sequences of the various forms of HD-5 peptides are shown in Table 1.

TABLE 1

| | SEQ ID | |
|---|---|---|
| HD-5 | NO: | AMINO ACID SEQUENCE |
| Pre- | SEQ ID | MRTIAILAAILLVALQAQAESLQERADEATTQK |
| pro- | NO:1 | QSGEDNQDLAISFAGNGLSALRTSGSQARATCY |
| peptide | | CRTG RCATRESLSGVCEISGRLYRLCCR |
| Pro- | SEQ ID | ESLQERADEATTQKQSGEDNQDLAISFAGNGLS |
| peptide | NO:2 | ALRTSGSQARATCYCRTGRCATRESLSG |
| | | VCEISGRLYRLCCR |
| Signal | SEQ ID | MRTIAILAAILLVALQAQA |
| peptide | NO:3 | |
| Mature | SEQ ID | ATCYCRTG RCATRESLSGVCEISGRLYRLCCR |
| peptide | NO:4 | |
| Pro piece | SEQ ID | ESLQERADEATTQKQSGEDNQDLAISFAGNGLS |
| | NO:5 | ALRTSGSQAR |
| Pro- | SEQ ID | ESLQERADEATTQKQSGEDNQDLAISFAGNGLS |
| HD-5^{Met61} | NO:6 | ALRTSGSQMRATCYCRTGRCATRESLSG |
| | | VCEISGRLYRLCCR |

If the mature HD-5 protein is allowed to be produced it can facilitate the destruction of attenuated bacteria which are being used as a vaccine. Thus, the attenuated bacteria do not have an opportunity to sufficiently stimulate the patient's immune system resulting in the generation of the desired amount of antibodies. Thus, any mechanism allowing for the inhibiting of the formation of the mature HD-5 protein, at least for a period of time, would facilitate the efficacy of vaccines which utilize attenuated bacteria. Alternatively, if the mature protein could be blocked by the attachment of molecules to it or the receptor surface on the cells to which the mature HD-5 protein attaches could be blocked, at least for a period of time, the efficacy of attenuated bacterial vaccines could be substantially enhanced. Further, by genetically engineering attenuated bacteria such that they are resistant to HD-5 it is possible to enhance the treatment of the patient by utilizing the effects of HD-5 on truly infectious bacteria while eliminating the undesirable effect of HD-5 on attenuated bacteria which are used to enhance the immune response needed in order to fight the infection.

For organizational purposes the aspects of the invention are provided in three groups as follows: (1) composition which comprises bacteria which contain defensin inhibitors which are expressed in the bacteria themselves (2) vaccine compositions of bacteria which are co-formulated with inhibitors of defensins (3) vaccine compositions of bacteria which are co-formulated with inhibitors of human pro-defensin processing. The three groups are further described in the following three sections:

Bacteria which are Resistant to Defensin Action in which the Resistance Determinant is Expressed in the Bacteria Themselves

An important aspect of this invention is a composition, comprising: a pharmaceutically acceptable excipient; and bacteria 1) which express an inhibitor of human defensin action, or 2) the bacteria are resistant after selection for HD-5 resistance by either spontaneous mutation, transposon mutagenesis, or chemical mutagenesis.

In one embodiment of this invention the bacteria are altered by a heterologous nucleotide, which may be operatively inserted into a plasmid or inserted into the genome, where the heterologous nucleotide encodes a defensin inhibitor protein or peptide. In a preferred embodiment, the heterologous nucleotide encodes a protein or peptide selected from the groups consisting of HD-5 peptide inhibitors such as HD-5 pro-piece (SEQ ID NO: 5) or Pro-HD-5^{Met61} (SEQ ID NO: 6). (see FIG. 2). In another embodiment of this invention, the bacteria are resistant to the antimicrobial actions of HD-5 after selection for HD-5 resistance by either spontaneous mutation, transposon mutagenesis, or chemical mutagenesis.

In one embodiment the bacteria are altered bacteria which are pathogenic in the unaltered state wherein the pathogenic bacteria are selected from the group consisting of Streptococcus, Listeria, Staphylococcus, Bacillus, Coryneforms, Enterobacteriaceae, Klebsiella, Serratia, Proteus, Shigella spp., Haemophilus, Non-typable Haemophilus influenza, Bordetella, Neisseria meningitidis, Pasteurella, Treponema. E. coli, Streptococcus pneumoniae, Helicobacter pylori, Vibrio cholerae, Yersinia spp., Porphyromonas gingivalis, Legionella pneumophila, Staphylococcus aureus, Clostridium botulinum, and Salmonella enterica.

In another embodiment, the bacteria are a Salmonella enterica bacteria selected from the group consisting of serovars Typhimurium, Enteritidis, Typhi, Abortus-ovi, Abortus-equi, Dublin, Gallinarum, and Pullorum.

Means for Attenuating Live Vaccines

The present invention may be used in connection with a variety of different vaccines. In one embodiment the invention is used with a live attenuated bacteria. The bacteria may be attenuated in any manner. The attenuation must result in a form of the bacteria such that the bacteria does not cause the patient to become ill when the vaccine is administered. One method of attenuation is described within published PCT application WO 00/45840 A1 which is incorporated herein by reference in its entirety along with the publications cited therein for their disclosure of methods of attenuation. One method of attenuation involves alteration of the activity of DNA adenine methylase (Dam). The Dam alteration methodology may include inhibiting the activity of the Dam gene or enhancing its activity relative to the activity of the wild type.

Pharmaceutical Compositions

Another important aspect of the invention is an immunogenic composition, comprising: a pharmaceutically acceptable excipient; and live attenuated bacteria, said bacteria which 1) express an inhibitor of human antimicrobial defensin action or 2) are inherently resistant, wherein the altered bacteria increases the immunogenicity of vaccine formulations against the bacteria or against heterologous antigens in human defensin transgenic mice, and higher species such as primates, and humans. The composition may comprise bacteria wherein the defensin inhibitory activity is encoded by a heterologous nucleotide, or the bacteria are resistant after selection for HD-5 resistance by either spontaneous mutation, transposon mutagenesis, or chemical mutagenesis.

Another important aspect of the invention is a method comprising the steps of: administering to a subject capable of generating an immune response a composition comprising a pharmaceutically acceptable excipient an immunogenic dose of altered bacteria wherein the defensin inhibitory activity is 1) encoded by a heterologous nucleotide, or 2) the bacteria are resistant after selection for HD-5 resistance by either spontaneous mutation, transposon mutagenesis, or chemical mutagenesis; and allowing the composition to remain in the subject for a time and under conditions to allow the subject to generate an immune response to the bacteria and produce antigen specific T cells or antibodies specific to the bacteria or heterologous antigen. The T cells and antibodies are highly specific for the bacteria or heterologous antigen which can be a human tumor antigen, a viral antigen, a bacterial antigen, a parasitic antigen.

In a preferred embodiment the method is carried out wherein an amount of antigen specific T cells or antibody produced by the subject exceeds 150% of an amount of T cells or antibody which would be produced by the subject administered altered bacteria 1) without the heterologous nucleotide which encodes a defensin inhibitor protein or peptide, or 2) the bacteria are resistant after selection for HD-5 resistance by either spontaneous mutation, transposon mutagenesis, or chemical mutagenesis.

Another important aspect of the invention is a method of eliciting an immune response in an individual, comprising: administering an immunogenic composition to an individual in an amount sufficient to elicit an immune response wherein the composition comprises a pharmaceutically acceptable carrier and a bacteria characterized by being resistant to the actions of human defensins, allowing the composition to remain in the individual for a time and under conditions to allow the individual to generate an immune response. The immune response is against a human tumor antigen, a viral antigen, a bacterial antigen, or a parasitic antigen.

Vaccine Compositions of Bacteria Co-Formulated with Inhibitors of Defensins

An important aspect of this invention is a composition, comprising: a pharmaceutically acceptable excipient; and bacteria which are co-formulated with inhibitors of defensins.

In one embodiment of this invention the attenuated vaccine bacteria are co-formulated with inhibitors of defensins, where the composition of the co-formulation is selected from the groups consisting of HD-5 peptide inhibitors such as HD-5 pro-piece (SEQ ID NO: 5) or Pro-HD-5^{met61} (SEQ ID NO: 6), serpins such as alpha 1-proteinase inhibitor or alpha 1-antichymotrypsin or derivatives; alpha 2-macroglobulin or derivatives; or a glycosaminoglycan such as dermatan sulfate.

In one embodiment the bacteria are altered bacteria which are pathogenic in the unaltered state wherein the pathogenic bacteria are selected from the group consisting of Streptococcus, Listeria, Staphylococcus, Bacillus, Coryneforms, Enterobacteriaceae, Klebsiella, Serratia, Proteus, Shigella spp., Haemophilus, Non Typable Haemophilus influenza, Bordetella, Neisseria meningitidis, Pasteurella, Treponema. E. coli, Streptococcus pneumoniae, Helicobacter pylori, Vibrio cholerae, Yersinia spp., Porphyromonas gingivalis, Legionella pneumophila, Staphylococcus aureus, Clostridium botulinum, and Salmonella enterica.

In one embodiment the bacteria are altered bacteria which are pathogenic in these unaltered state wherein the pathogenic bacteria are selected from the group consisting of Salmonella enterica. In another embodiment, the bacteria are a Salmonella enterica bacteria selected from the group consisting of serovars Typhimurium, Enteritidis, Typhi, Abortus-ovi, Abortus-equi, Dublin, Gallinarum, and Pullorum.

Another important aspect of the invention is an immunogenic composition, comprising: a pharmaceutically acceptable excipient; and live bacteria, said bacteria which are co-formulated with inhibitors of defensins, wherein the altered bacteria increases the immunogenicity of vaccine formulations against heterologous antigens in human defensin transgenic mice, primates, and humans. The composition may comprise bacteria which are co-formulated with inhibitors of defensins where the composition of the co-formulation is selected from the groups consisting of HD-5 peptide inhibitors such as HD-5 pro-piece or Pro-HD-5^{Met61} (see FIG. 2), serpin such as alpha 1-proteinase inhibitor or alpha 1-antichymotrypsin or derivatives; alpha 2-macroglobulin or derivatives; or a glycosaminoglycan such as dermatan sulfate.

Another important aspect of the invention is a method comprising the steps of: administering to a subject capable of generating an immune response a composition comprising a pharmaceutically acceptable excipient an immunogenic dose of bacteria where the co-formulation is selected from the groups consisting of HD-5 peptide inhibitors such as HD-5 pro-piece or Pro-HD-5^{Met61}; serpin such as alpha 1-proteinase inhibitor or alpha 1-antichymotrypsin or derivatives; alpha 2-macroglobulin or derivatives; or a glycosaminoglycan such as dermatan sulfate and allowing the composition to remain in the subject for a time and under conditions to allow the subject to generate an immune response to the bacteria and produce antigen specific T cells or antibodies specific to the bacteria or heterologous antigen. The T cells and antibodies are highly specific for the bacteria or heterologous antigen which can be a human tumor antigen, a viral antigen, a bacterial antigen, or a parasitic antigen.

In a preferred embodiment the method is carried out wherein an amount antigen specific T cells or antibody produced by the subject exceeds 150% of an amount of T cells or antibody which would be produced by the subject administered altered bacteria without the co-formulation which is defensin inhibitor.

Another important aspect of the invention is a method of eliciting an immune response in an individual, comprising: administering an immunogenic composition to an individual in an amount sufficient to elicit an immune response, wherein the composition comprises a pharmaceutically acceptable carrier and a bacteria co-formulated with inhibitors of defensins, characterized by a being resistant to the actions of human defensins, allowing the composition to remain in the individual for a time and under conditions to allow the individual to generate an immune response. The immune response is against a human tumor antigen, a viral antigen, a bacterial antigen, or a parasitic antigen.

Vaccine Compositions of Bacteria which are Co-Formulated with Inhibitors of Human Pro-Defensin Processing

An important aspect of this invention is a composition, comprising: a pharmaceutically acceptable excipient; and bacteria which are co-formulated with inhibitors of pro-defensin processing.

In one embodiment of this invention the bacteria are co-formulated with inhibitors of pro-defensin processing, where the composition of the co-formulation is selected from the groups consisting of trypsin inhibitors such as 4-amidinophelylmethane sulfonyl-fluoride (APMSF), aprotinin or soya bean trypsin inhibitor.

In one embodiment the bacteria are altered bacteria which are pathogenic in the unaltered state wherein the pathogenic bacteria are selected from the group consisting of Streptococcus, Listeria, Staphylococcus, Bacillus, Coryneforms, Enterobacteriaceae, Klebsiella, Serratia, Proteus, Shigella spp., Haemophilus, Non Typable Haemophilus influenza, Bordetella, Neisseria meningitidis, Pasteurella, Treponema. E. colt, Streptococcus pneumoniae, Helicobacter pylori, Vibrio cholerae, Yersinia spp., Porphyromonas gingivalis, Legionella pneumophila, Staphylococcus aureus, Clostridium botulinum, and Salmonella enterica.

In one embodiment the bacteria are altered bacteria which are pathogenic in these unaltered state wherein the pathogenic bacteria are selected from the group consisting of Salmonella enterica bacteria selected from the group consisting of serovars Typhimurium, Enteritidis, Typhi, Abortus-ovi, Abortus-equi, Dublin, Gallinarum, and Pullorum.

Another important aspect of the invention is an immunogenic composition, comprising: a pharmaceutically acceptable excipient; and live bacteria, said bacteria which are co-formulated with inhibitors of pro-defensin processing, wherein the altered bacteria increases the immunogenicity of vaccine formulations against the bacteria or against heterologous antigens in human defensin transgenic mice, primates, and humans. The composition may comprise bacteria which are co-formulated with inhibitors of defensin processing where the composition of the co-formulation is selected from the groups consisting of trypsin inhibitors such as APMSF, aprotinin or soya bean trypsin inhibitor.

Another important aspect of the invention is a method comprising the steps of: administering to a subject capable of generating an immune response a composition comprising a pharmaceutically acceptable excipient an immunogenic dose of bacteria where the co-formulation is selected from the groups consisting of trypsin inhibitors such as APMSF, aprotinin or soya bean trypsin inhibitor; and allowing the composition to remain in the subject for a time and under conditions to allow the subject to generate an immune response to the bacteria and produce antigen specific T cells or antibodies specific to the bacteria or heterologous antigen. The T cells and antibodies are highly specific for the bacteria or heterologous antigen which can be a human tumor antigen, a viral antigen, an bacterial antigen, a parasitic antigen.

In a preferred embodiment the method is carried out wherein an amount antigen specific T cells or antibody produced by the subject exceeds 150% of an amount of T cells or antibody which would be produced by the subject administered altered bacteria without the co-formulation which is an inhibitor of defensin processing.

Another important aspect of the invention is a method of eliciting an immune response in an individual, comprising: administering an immunogenic composition to an individual in an amount sufficient to elicit an immune response, wherein the composition comprises a pharmaceutically acceptable carrier and a bacteria co-formulated with inhibitors of pro-defensin processing, allowing the composition to remain in the individual for a time and under conditions to allow the individual to generate an immune response. The immune response is a human tumor antigen, a viral antigen, a bacterial antigen, or a parasitic antigen.

Effects of Inhibiting HD-5

HD-5 inhibitors enhance efficacy of whole organism vaccines such as Salmonella enterica serovar Typhimurium. Human defensin 5 (HD-5), is the major defensin in humans which is bactericidal for Salmonella typhimurium infection. HD-5 transgenic mice are dramatically more resistant to Salmonella typhimurium infection providing a model system to study effectiveness of Salmonella typhimurium as a platform vaccine carrier in the presence of inhibitors of HD-5. See Gosh D. et al., Nature Immunology, 3:583 (2002); Zasloff M, Nature Immunology, 3:508 (2002). HD-5 inhibitors increase efficacy of the Salmonella enterica based human CEA-DNA vaccine. Human CEA (carcinoembryonic antigen) is a tumor antigen for which protective immunity can be induced by vaccination with a live attenuated Salmonella enterica vaccine. See Xiang R. et al., Clinical Cancer Research, 7: 856s (2001). Efficacy of the vaccine is measured by conducting cytotoxicity (CTL) assays in HD-5 transgenic mice to measure immune response to the human CEA antigen. CTL assays are conducted in mice immunized orally with various doses of the vaccine (RemeStim-CEA), with or without addition of the HD-5 inhibitor. The Pro-piece of HD-5, proHD-5^{Met61} (a proHD-5 containing a mutation that changes an arginine to a methionine which inhibits processing to mature peptide), and proteins such as alpha 1-proteinase inhibitor and alpha 1-antichymotrypsin or alpha 2-macroglobulin inhibit the bactericidal activity of the mature, active, defensin. Inhibition tests of mature defensin anti-bacterial activity against Salmonella enterica serovar Typhimurium are conducted in vitro, by incubation of the bacterial strain with HD-5 with or without addition of the various HD-5 inhibitors, followed by plating on media and counting number of viable colonies (CFU). See Valore E V, et al. J Clin Invest, 97:1624 (1996); Panyutich A, et al., Am J Respir Cell Mol Biol., 12:351 (1995); Panyutich A, Ganz T, Am J Respir Cell Mol Biol., 5:101 (1991); Gosh D. et al., Nature Immunology, 3:583 (2002).

Proteoglycans are glycosaminoglycan (GAG)-containing molecules characterized by core protein and type of associated GAG. The GAG side chains bound to the core protein may be chondroitin sulfate, dermatan sulfate, heparan sulfate (HS), heparin, or keratan sulfate. The glycosaminoglycan dermatan sulphate enhances the effectiveness of the Salmonella enterica serovar Typhimurium based CEA-DNA vaccine as measured by cytotoxicity assays. Dermatan sulfate inactivates alpha-defensin anti-bacterial activity against Salmonella enterica serovar Typhimurium in vitro. See, Linhardt R J, Hileman R E, Gen Pharmacol, 26:443 (1995); Schmidtchen A, et al., Mol Microbiol, 39:708 (2001).

Bacterial production and secretion of the defensin HD-5 pro-piece inhibits mature defensin HD-5 and increase the efficacy of attenuated Salmonella enterica based vaccines. Local production and secretion of the pro piece (amino acids 20-62) by the Salmonella enterica based vaccine enhances the effectiveness of the live attenuated bacterial vaccine, since the pro-peptides from defensins inhibit the bactericidal activity of the mature, active, defensins. See Valore E V, et al. J Clin Invest, 97:1624 (1996). Local production and secretion of the pro-piece is achieved by cloning the cDNA sequence encoding the pro-domain of human HD-5 (amino acids 20-62) in a prokaryotic expression system that targets the recombinant protein for secretion. The secretion system is the E. coli alpha-hemolysin secretion system, which has previously been shown to express and secrete heterologous proteins in Salmonella enterica. The pro-piece is expressed as a fusion protein with a hemaglutinnin (HA) tag epitope followed by sequences from the HlyA bacterial protein which targets the pro-piece for secretion. See Gentschev I, et al., Trends in Microbiology, 10:39 (2002); Tzchaschel, B D, et al., Nat Biotechnology, 14:765 (1996). The pro-piece of HD-5 is presumed to be locally available at the site of HD-5 exposure, to bind to the mature HD-5 molecule and inhibit its bactericidal activity. Expression of the pro-piece is detected by Western Blot analyses using the HA-tag specific antibody.

Inhibition of mature defensin anti-bacterial activity against Salmonella typhimurium assays are conducted in vitro, by incubation with the mature HD-5 and the bacterial strain expressing the pro-piece of HD-5 compared to bacteria expressing an unrelated peptide, followed by plating on media and counting number of viable colonies (CFU). See Valore E V, et al. J Clin Invest, 97:1624 (1996); Panyutich A, et al., Am J Respir Cell Mol Biol., 12:351 (1995); Panyutich A, Ganz T, Am J Respir Cell Mol Biol., 5:101 (1991); Gosh D. et al., Nature Immunology, 3:583 (2002).

Immunization with the vaccine strain expressing the pro-piece of HD-5 enhances the effectiveness of the S. typhimurium based CEA-DNA vaccine in HD-5 transgenic mice as measured by cytotoxicity assays.

Inhibitors of human pro-defensin processing increase the efficacy of the attenuated Salmonella enterica vaccine. Processing of pro-HD-5 in the small intestine is mediated by trypsin which is co-localized with pro-HD-5 in the Paneth cells granules. HD-5 is proposed to be proteolytically processed after secretion. Inhibitors of trypsin, such as APMSF, inhibit maturation of pro-HD-5 to mature HD-5 by trypsin in vitro. Oral immunization of HD-5 transgenic mice with the Salmonella vaccine strain increases the efficacy of the vaccine as measured by testing increased immunity to the human CEA antigen. Similar results are obtained with other trypsin inhibitors, aprotinin and soya bean trypsin inhibitor. See Gosh D. et al., Nature Immunology, 3:583 (2002); Cole T C, et al. Biochim Biophys Acta, 990:254 (1989); Laura R, et al., Biochemistry, 19:4859 (1980); Azougagh Oualane F, et al. Thromb Res, 68:185 (1992); Uchino T, et al. J Biol. Chem. 1268:527 (1993); Sweadner K J, Anal Biochem 194:130 (1991).

Human-defensin 5 (HD-5)-resistant mutants of Salmonella enterica increase the efficacy of Salmonella enterica based vaccines: A mechanism for the vaccine strain to avoid the microbicidal effects of HD-5 is provided by selecting for HD-5-resistant mutants. Such mutant has a genomic mutation that provides ability to resist or circumvent the killing activity of HD-5. Transposon mutagenesis is a well-characterized method for obtaining mutations in bacteria, see Eisenstein, I, et. al., Vaccine 16: 24 (1998). This method leads to isolation of mutations in S. enterica which allow the bacteria to survive a lethal exposure to HD-5 in liquid medium. Such assays have been used previously to demonstrate the bactericidal effects of HD-5 on S. enterica, see Ghosh et. al., Nature Immunology 3 583 (2002).

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Handbook of Experimental Immunology (D. M. Wei & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction (Mullis et al., eds., 1994); Current Protocols in Immunology (J. E. Coligan et al., eds., 1991); Short Protocols in Molecular Biology (Wiley & Sons, 1999).

Compositions of the Invention

The compositions described are useful for eliciting an immune response, and/or treating or preventing disease, such as cancer, and viral, bacterial, parasitic, or fingal infections. The live vaccines produced herein may serve as carriers for antigens, such as immunogens of cancer cells or pathogens thereby producing a multiple immunogenic response.

The subject invention is particularly applicable to a wide variety of bacteria such as Salmonella enterica.; as well as others which are known or may be discovered to cause infections in mammals.

Preferably, the compositions comprise a pharmaceutically acceptable excipient. A pharmaceutically acceptable excipient is a relatively inert substance that facilitates administration of a pharmacologically effective substance. For example, an excipient can give form or consistency to the vaccine composition, or act as a diluent. Suitable excipients include but are not limited to stabilizing agents, wetting and emulsifying agents, salts for varying osmolarity, encapsulating agents, buffers, and skin penetration enhancers. Examples of pharmaceutically acceptable excipients are described in Remington's Pharmaceutical Sciences (Alfonso R. Gennaro, ed., 19th edition, 1995).

The vaccines can be used with a wide variety of domestic animals, as well as humans. Included among domestic animals which are treated by vaccines today or could be treated, if susceptible to bacterial diseases, are chickens, cows, pigs, horses, goats, and sheep, to name the more important domestic animals.

The invention provides live vaccines which may be used as vectors or carriers for an antigen. The antigen may be any antigen, including an antigen of a tumor cell, virus, fungi, parasite, bacteria, or immune disease antigen. The antigen may be added as an admixture, attached or associated with the bacteria, or one or more structural genes coding for the desired antigen(s) may be introduced into the non-virulent pathogenic vaccine as an expression cassette. Accordingly, any of the mutant bacteria described for use in the vaccines described herein may further comprise an expression cassette having one or more structural genes coding for a desired antigen. The expression cassette comprises the structural gene or genes of interest under the regulatory control of the transcriptional and translational initiation and termination regions which naturally border the structural gene of interest or which are heterologous with respect to the structural gene. Where bacterial or bacteriophage structural genes are involved, the natural or wild-type regulatory regions will usually, but not always, suffice. It may be necessary to join regulatory regions recognized by the non-virulent pathogen to structural genes for antigens isolated from eukaryotes and occasionally prokaryotes.

The expression cassette may be a recombinant construct or may be, or form part of, a naturally occurring plasmid. If the expression cassette is a recombinant construct, it may be joined to a replication system for episomal maintenance or it may be introduced into the non-virulent pathogenic bacteria under conditions for recombination and integration into the non-virulent pathogen's chromosomal DNA. Structural genes for antigens of interest may encode tumor antigens such as carcinoembryonic antigen, viral proteins such as human papilloma virus, or enzyme pathways such as those involved in synthesis of carbohydrate antigens such as lipopolysaccharide (LPS). For example, among the antigens expressed in other live attenuated Salmonella vaccines are Fragment C of tetanus toxin, the B subunit of cholera toxin, the hepatitis B surface antigen, and Vibrio cholerae LPS. Additionally, the HIV antigens GP120 and GAG have been expressed in attenuated Mycobacterium bovis BCG and Shigella soneii LPS has been expressed in attenuated Vibrio cholerae. The construct or vector may be introduced into the host strain through a number of well known methods such as, transduction, conjugation, transformation, electroporation, transfection, etc.

The immunogenic compositions described herein may be used with an adjuvant which enhances the immune response against the pathogenic bacteria such as Salmonella enterica. Adjuvants are especially suitable for killed vaccines, but need not be limited to this use. Suitable adjuvants are known in the art and include aluminum hydroxide, alum, QS-21 (U.S. Pat. No. 5,057,540), DHEA (U.S. Pat. Nos. 5,407,684 and 5,077,284) and its derivatives and precursors, e.g., DHEA-S, beta-2 microglobulin (WO 91/16924), muramyl dipeptides, muramyl tripeptides (U.S. Pat. No. 5,171,568) and monophosphoryl lipid A (U.S. Pat. No. 4,436,728; WO 92/16231) and its derivatives, e.g., DETOX™, and BCG (U.S. Pat. No. 4,726,947). Other suitable adjuvants include, but are not limited to, aluminum salts, squalene mixtures (SAF-1), muramyl peptide, saponin derivatives, mycobacterium wall preparations, mycolic acid derivatives, nonionic block copolymer surfactants, Quil A, cholera toxin B subunit, polyphosphazene and derivatives, and immunostimulating complexes (ISCOMs) such as those described by Takahashi et al. (1990) Nature 344:873-875. For veterinary use and for production of antibodies in animals, mitogenic components of Freund's adjuvant can be used. The choice of an adjuvant will depend in part on the stability of the vaccine in the presence of the adjuvant, the route of administration, and the regulatory acceptability of the adjuvant, particularly when intended for human use. For instance, alum is approved by the United States Food and Drug Administration (FDA) for use as an adjuvant in humans.

In some embodiments, the immunogenic composition may also comprise a carrier molecule (with or without an adjuvant). Carriers are known in the art. Pltokin, Vaccines 3rd Ed. Philadelphia, WB Saunders Co. (1999). Bacterial carriers (i.e., carriers derived from bacteria) include, but are not limited to, cholera toxin B subunit (CTB); diphtheria toxin mutant (CRM197); diphtheria-toxoid; group B streptococcus alpha C protein; meningococcal outer membrane protein (OMPC); tetanus toxoid; outer membrane protein of non-typeable Haemophilus influenzae (such as P6); recombinant class 3 porin (rPorBP of group B. meningococci; heat-killed Burcella abortus; heat-killed Listeria monocytogeneis; and Pseudomonas aeruginosa recombinant exoprotein A. Another carrier is keyhole limpet hemocyanin (KLH).

The vaccines of the present invention are suitable for or oral pills, solutions or suspensions, oil in water or water in oil emulsions and the like, Administration can also be, intranasal, intrapulmonary (i.e., by aerosol), intravaginal, or intrarectal. Additional formulations which are suitable for other modes of administration include suppositories . The route of administration will depend upon the condition of the individual and the desired clinical effect.

The subject vaccines may be used in a wide variety of vertebrates. The subject vaccines will find particular use with mammals, such as man, and domestic animals. Domestic animals include bovine, ovine, porcine, equine, caprine, domestic fowl, Leporidate e.g., rabbits, or other animals which may be held in captivity or may be a vector for a disease affecting a domestic vertebrate. The manner of application of the may be varied widely, any of the conventional methods for administering being applicable. These include oral application, on a solid physiologically acceptable base or in a physiologically acceptable dispersion. The dosage of the vaccine will depend inter alia on route of administration and will vary according to the species to be protected. One or more additional administrations may be provided as booster doses.

Kits and Strains

The invention also provides attenuated bacterial strains as described herein. Preferred strains are Salmonella enterica strains.

The present invention also encompasses kits containing any one or more of the strains and/or vaccine formulations described herein in suitable packaging. The kit may optionally provide instructions, such as for administration to effect any one or more of the following: eliciting an immune response; treatment of infection; prevention of infection; amelioration of one or more symptoms of infection. In some embodiments, the instructions are for administration to a non-human, such as chicken, cattle, pigs, or other farm animal. In other embodiments, the instruction are for administration to a human.

Methods of the Invention

The invention also provides methods using the immunogenic compositions described herein, screening methods to identify potentially useful agents which are resistant to the antimicrobial action of human defensin, as well as methods of preparing the immunogenic compositions described herein.

With respect to any methods involving administration of any of the compositions described herein, it is understood that any one or more of the compositions can be administered, i.e., the compositions can be administered alone or in combination with each other. Further, the compositions can be used alone or in conjunction with other modalities (i.e., clinical intervention), for the purpose of prevention and/or treatment.

Use of Immunogenic Compositions for Eliciting an Immune Response, Prevention of and Treating Disease

In some embodiments, the invention provides methods using the immunogenic compositions described herein to elicit an immune response in an individual. Generally, these methods comprise administering any one or more of the immunogenic compositions described herein to an individual in an amount sufficient to elicit an immune response. The immune response is against the bacteria themselves or against a heterologous antigen

The immune response may be a B cell and/or T cell response. Preferably, the response is antigen-specific, i.e., the response is against the bacteria used in the immunogenic composition (i.e., a response against an antigen associated with the bacteria used is detected) or against a heterologous antigen

Preferably, the immune response persists in the absence of the vaccine components. Accordingly, in some embodiments, the immune response persists for about any of the following after administration of an immunogenic composition described herein (if given as multiple administrations, preferably after the most recent administration): four weeks, six weeks, eight weeks, three months, four months, six months, and yearly.

In order to determine the effect of administration of an immunogenic composition described herein, the individual may be monitored for either an antibody (humoral) or cellular immune response against the bacteria, or a combination thereof, using standard techniques in the art.

Suitable individuals for receiving the compositions have been described above and likewise apply to these methods. Generally, such individuals display a symptom and/or disease state, or are at risk for the disease state.

The vaccines are administered in a manner compatible with the dosage formulation, and in such amount as will be therapeutically effective. The quantity to be administered depends on the individual to be treated, the capacity of the individual's immune system to generate an immune response, the route of administration, and the degree of protection desired. Precise amounts of active ingredient required to be administered may depend on the judgment of the practitioner in charge of treatment and may be peculiar to the individual.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An immunogenic composition comprising:
a pharmaceutically acceptable excipient; and
an attenuated bacteria which is resistant to an antimicrobial action of a human defensin.

2. The composition of claim 1, wherein the attenuated bacteria expresses an inhibitor of a human defensin.

3. The composition of claim 2, wherein the human defensin is HD-5.

4. The composition of claim 3, wherein the attenuated bacteria expresses a HD-5 peptide inhibitor selected from the group consisting of HD-5 pro-piece (SEQ ID NO: 5) and pro-HD-5^{Met61} SEQ ID NO: 6).

5. The composition of claim 1, wherein the attenuated bacteria is selected for resistance to a human defensin by a process selected from the group consisting of spontaneous mutation, transposon mutagenesis and chemical mutagenesis.

6. The composition of claim 1, wherein the attenuated bacteria is Salmonella enterica selected from the group consisting of serovars Typhimurium, Enteritidis, Typhi, Abortus-ovi, Abortus-equi, Dublin, Gallinarum, and Pullorum.

7. The composition of claim 1, wherein the attenuated bacteria is an attenuated pathogenic bacteria selected from the group consisting of Streptococcus, Listeria, Staphylococcus, Bacillus, Coryneforms, Enterobacteriaceae, Klebsiella, Serratia, Proteus, Shigella spp., Haemophilus, Non-Typable Haemophilus influenza, Bordetella, Neisseria meningitidis, Pasteurella, Treponem, E. coli, Streptococcus pneumoniae, Helicobacter pylori, Vibrio cholerae, Yersinia spp., Porphyromonas gingivalis, Legionella pneumophila, Staphylococcus aureus, Clostridium botulinum, and Salmonella enterica.

8. The composition of claim 1, wherein the bacteria is genetically engineered to express an antigen selected from the group consisting of a human tumor antigen, a viral antigen, a bacterial antigen, a fungal antigen, a parasitic antigen, and an immune disease antigen.

9. The composition of claim 1, wherein the composition is an oral formulation.

10. An immunogenic composition comprising:
a pharmaceutically acceptable excipient;
an attenuated bacteria; and
at least one inhibitor of a human defensin.

11. The immunogenic composition of claim 10, wherein the attenuated bacteria expresses an inhibitor of a human defensin.

12. The immunogenic composition of claim 11, wherein the human defensin is HD-5.

13. The immunogenic composition of claim 10, wherein the inhibitor of human defensin is selected from the group consisting of HD-5 pro-piece (SEQ ID NO: 5), Pro-HD-5^{Met61}(SEQ ID NO: 6), a serpin, alpha 1-proteinase inhibitor, alpha 1-antichymotrypsin and derivatives thereof, alpha 2-macroglobulin and derivatives thereof, a glycosaminoglycan, and dermatan sulfate.

14. The immunogenic composition of claim 10, wherein the attenuated bacteria is Salmonella enterica selected from the group consisting of serovars Typhimurium, Enteritidis, Typhi, Abortus-ovi, Abortus-equi, Dublin, Gallinarum, and Pullorum.

15. The immunogenic composition of claim 10, wherein the attenuated bacteria is an attenuated pathogenic bacteria selected from the group consisting of Streptococcus, Listeria, Staphylococcus, Bacillus, Coryneforms, Enterobacteriaceae, Klebsiella, Serratia, Proteus, Shigella spp., Haemophilus, Non-Typable Haemophilus influenza, Bordetella, Neisseria men ingitidis, Pasteurella, Trepon em, E. coli, Streptococcus pneumoniae, Helicobacter pylori, Vibrio cholerae, Yersinia spp., Porphyromonas gingivalis, Legionella pneumophila, Staphylococcus aureus, Clostridium botulinum, and Salmonella enterica.

16. The immunogenic composition of claim 10, wherein the bacteria is genetically engineered to express an antigen selected from the group consisting of a human tumor antigen, a viral antigen, a bacterial antigen, a fungal antigen, a parasitic antigen, and an immune disease antigen.

17. The immunogenic composition of claim 10, wherein the bacteria is attenuated by alteration in its Dam gene.

18. The immunogenic composition of claim 17, wherein expression of the Dam gene is increased or decreased relative to wild type.

19. The immunogenic composition of claim 10, wherein the inhibitor of a human defensin inhibits processing of a human defensin pro-peptide.

20. The immunogenic composition of claim 19, wherein the inhibitor of human defensin pro-peptide is selected from the group consisting of a trypsin inhibitor, 4-amidinophelylmethane sulfonyl-fluoride (APMSF), aprotinin and soybean trypsin inhibitor.

21. The immunogenic composition of claim 19, wherein the human defensin is HD-5.

22. The immunogenic composition of claim 20, wherein the inhibitor of human defensin pro-peptide is selected from the group consisting of HD-5 pro-piece (SEQ ID NO: 5) and pro-HD-5^{Met61} (SEQ ID NO: 6).

23. The immunogenic composition of claim 19, wherein the attenuated bacteria is Salmonella enterica selected from the group consisting of serovars Typhimurium, Enteritidis, Typhi, Abortus-ovi, Abortus-equi, Dublin, Gallinarum, and Pullorum.

24. The immunogenic composition of claim 19, wherein the attenuated bacteria is an attenuated pathogenic bacteria selected from the group consisting of Streptococcus, Listeria, Staphylococcus, Bacillus, Coryneforms, Enterobacteriaceae, Klebsiella, Serratia, Proteus, Shigella spp., Haemophilus, Non-Typable Haemophilus influenza, Bordetella, Neisseria meningitidis, Pasteurella, Treponem, E. coli, Streptococcus pneumoniae, Helicobacterpylori, Vibrio cholerae, Yersinia spp., Porphyromonas gingivalis, Legionella pneumophila, Staphylococcus aureus, Clostridium botulinum, and Salmonella enterica.

25. The immunogenic composition of claim 19, wherein the bacteria is genetically engineered to express an antigen selected from the group consisting of a human tumor antigen, a viral antigen, a bacterial antigen, a fungal antigen, a parasitic antigen, and an immune disease antigen.

26. The immunogenic composition of claim 19, wherein the bacteria is attenuated by alteration in its Dam gene.

27. The immunogenic composition of claim 26, wherein expression of the Dam gene is increased or decreased relative to wild type.

28. A bacteria comprising:
(a) a genetic modification resulting in altered expression of DNA adenine methylase (Dam) relative to the wild type sufficient to attenuate the bacteria's virulence; and
(b) a resistance to human-defensin 5 (HD-5) sufficient to allow the bacteria to remain in a patient for a period of time sufficient to generate an immune response.

29. The bacteria of claim 28, wherein the bacteria is selected from the group consisting of Streptococcus, Listeria, Staphylococcus, Bacillus, Coryneforms, Enterobacteriaceae, Klebsiella, Serratia, Proteus, Shigella spp., Haemophilus, Non-Typable Haemophilus influenza, Bordetella, Neisseria meningitidis, Pasteurella, Treponema, E. coli, Streptococcus pneumoniae, Helicobacterpylori, Vibrio cholerae, Yersinia spp., Porphyromonas gingivalis, Legionella pneumophila, Staphylococcus aureus, Clostridium botulinum, and Salmonella enterica.

30. The bacteria of claim 29, wherein the Salmonella enterica is chosen from serovars Typhimurium, Enteritidis, Typhi, Abortus-ovi, Abortus-equi, Dublin, Gallinarum, and Pullorum.

31. The bacteria of claim 28, further comprising a second genetic modification resulting in expression of an antigen selected from the group consisting of a human tumor antigen, a viral antigen, a bacterial antigen, a fungal antigen, a parasitic antigen, and an immune disease antigen.

32. A method of eliciting an immune response in an individual, comprising:
administering an immunogenic composition to an individual in an amount sufficient to elicit an immune response, wherein the composition comprises a pharmaceutically acceptable carrier, and a live attenuated bacteria resistant to human defensins;
allowing the composition to remain in the individual for a time and under conditions such that the individual generates an immune response.

33. The method of claim 32, wherein the live attenuated bacteria expresses a surface antigen selected from the group consisting of a human tumor antigen, a viral antigen, a bacterial antigen, a fungal antigen, a parasitic antigen, and an immune disease antigen.

34. The method of claim 32, wherein the bacteria is selected from the group consisting of Streptococcus, Listeria, Staphylococcus, Bacillus, Coryneforms, Enterobacteriaceae, Klebsiella, Serratia, Proteus, Shigella spp., Haemophilus, Non-Typable Haemophilus influenza, Bordetella, Neisseria meningitidis, Pasteurella, Treponema, E. coli, Streptococcus pneumoniae, Helicobacterpylori, Vibrio cholerae, Yersinia spp., Porphyromonas gingivalis, Legionella pneumophila, Staphylococcus aureus, Clostridium botulinum, and Salmonella enterica.

35. The method of claim 34, wherein the Salmonella enterica is chosen from serovars Typhimurium, Enteritidis, Typhi, Abortus-ovi, Abortus-equi, Dublin, Gallinarum, and Pullorum.

36. The method of claim 32, wherein the immunogenic composition is an oral formulation.
